(19) United States
(12) Patent Application Publication
(10) Pub. No.: US 2015/0157588 A1
(43) Pub. Date: Jun. 11, 2015

(54) Pharmaceutical Composition Comprising Amino-Phenyl-Acetic Acid Octadec-(Z)-9-enyl Ester and Use Thereof for Treating Tumors

(71) Applicant: Yeda Research and Development Co., Ltd., Rehovot (IL)

(72) Inventors: Irun R. Cohen, Rehovot (IL); Meir Shinitzky, Rehovot (IL); Raanan Margalit, Rehovot (IL); Michal Cohen-Sfady, Rehovot (IL); Alexandra Zanin-Zhorov, Rehovot (IL)

(21) Appl. No.: 14/412,838

(22) PCT Filed: Jul. 4, 2013
(86) PCT No.: PCT/IL2013/050573

§ 371 Date: Jan. 5, 2015

Related U.S. Application Data
(60) Provisional application No. 61/668,230, filed on Jul. 5, 2012.

(51) Int. Cl. A61K 31/216

(52) U.S. Cl. CPC A61K 31/216

(57) ABSTRACT

The present invention relates to the compound amino-phenyl-acetic acid octadec-(Z)-9-enyl ester, an enantiomer thereof or a pharmaceutically acceptable salt thereof or a pharmaceutical composition comprising it, for use in the treatment of tumors and metastases, and to methods for treating a tumor or metastases comprising administering said compound to a subject in need thereof. The present invention particularly relates to said compound when it is dissolved in an ethanol solution.

### FIELD OF THE INVENTION

The present invention relates to pharmaceutical compositions for treatment of tumors.

### BACKGROUND OF THE INVENTION

WO/2004/032824, by the same applicant, discloses esters of long-chain fatty alcohols with carboxylic acids containing at least one basic group that can act as anti-inflammatory immunomodulators and can be used for the treatment of inflammation, particularly immunologically-mediated inflammation, and as adjuvants in combination with specific antigens involved in both cellular and humoral responses, wherein said adjuvant serves as a carrier, or as depot or as immune potentiator/enhancer. Some of these esters, including amino-phenyl-acetic acid octadec-(Z)-9-enyl ester were described as novel compounds.

WO/2008/106092 discloses enantiomers of amino-phenyl-acetic acid octadec-(Z)-9-enyl ester and shows that both enantiomers were able to inhibit inflammation. Inflammation has been recently shown to be associated with the development and progression of tumors (Karin M., Inflammation and cancer: the long reach of Ras., 2005, Nature Medicine 11:20-21).

### SUMMARY OF THE INVENTION

It has been found, in accordance with the present invention, that amino-phenyl-acetic acid octadec-(Z)-9-enyl ester of formula I hereinbelow:

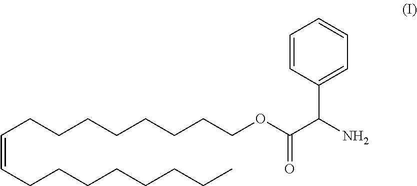 [SMILES: CCCCCCCC/C=C\CCCCCCCCOC(=O)C(N)c1ccccc1]

has an anti-tumor effect, in addition to its known anti-inflammatory effect.

It has further been found that when dissolved in ethanol solution, amino-phenyl-acetic acid octadec-(Z)-9-enyl ester manifests different biological effects on many genes including some involved in apoptosis and cell cycle than those caused by amino-phenyl-acetic acid octadec-(Z)-9-enyl ester dissolved in an aqueous vehicle. Subsequently, it has been shown in accordance with the presence invention that amino-phenyl-acetic acid octadec-(Z)-9-enyl ester dissolved in ethanol solution is a very effective anti-tumor agent.

The enantiomers R and S of the compound of Formula I above have the following structural formulas Ia (R) and Ib (S):

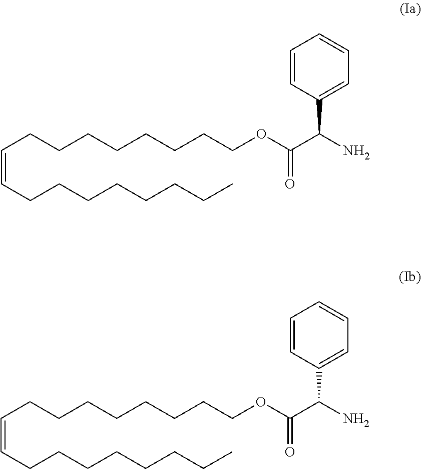 [SMILES: CCCCCCCC/C=C\CCCCCCCCOC(=O)[C@H](N)c1ccccc1 ; CCCCCCCC/C=C\CCCCCCCCOC(=O)[C@@H](N)c1ccccc1]

The present invention relates to amino-phenyl-acetic acid octadec-(Z)-9-enyl ester or an R or S enantiomer thereof or a pharmaceutically acceptable salt thereof, for use in treatment of tumors and metastases.

The present invention further relates to a pharmaceutical composition for treatment of tumors and metastases, comprising amino-phenyl-acetic acid octadec-(Z)-9-enyl ester, an enantiomer thereof or pharmaceutically acceptable salts thereof and a pharmaceutically acceptably carrier.

The present invention relates still further to methods of treating tumors and metastases in a subject in need thereof, comprising administering to the subject a therapeutically effective amount of amino-phenyl-acetic acid octadec-(Z)-9-enyl ester, an enantiomer thereof or a pharmaceutically acceptable salt thereof.

The present invention relates yet further to a method for enhancing apoptosis in a tumor or metastases, comprising administering to an individual in need thereof a therapeutically effective amount of amino-phenyl-acetic acid octadec-(Z)-9-enyl ester, an enantiomer thereof or a pharmaceutically acceptable salt thereof, dissolved in ethanol solution, thus enhancing apoptosis of said tumor or metastases in said individual.

### BRIEF DESCRIPTION OF DRAWINGS

FIG. 1 shows the day after tumor injection on which the indicated numbers of mice reached a tumor size of 8×8 mm and were resected. Mice were injected with 3LL tumor cells and treated subcutaneously with 100 μg of amino-phenyl-acetic acid octadec-(Z)-9-enyl ester in Phosphate Buffered Saline (PBS) injected twice weekly, starting from day 6 following tumor injection (black bars) or left untreated (white bars).

FIG. 2 shows mortality of mice from lung metastasis after excision of tumors. Mice were injected with 3LL tumor cells, and treated subcutaneously with 100 μg of amino-phenyl-acetic acid octadec-(Z)-9-enyl ester in PBS injected twice weekly either from day 6 after tumor injection (circles) or from the time of excision of the tumor, after reaching a size of 8 mm×8 mm (squares), or left untreated (triangles).

FIG. 3 shows that amino-phenyl-acetic acid octadec-(Z)-9-enyl ester in ethanol solution induced massive apoptosis of the Jurkat transformed T cell line. % apoptosis was measured by Fluorescence Activated Cell Sorter (FACS) analysis with a hypo-diploid nuclei propidium iodide (PI) staining. White bars (left bar of each pair) represent cells treated with 10 μg of amino-phenyl-acetic acid octadec-(Z)-9-enyl ester in ethanol solution, and black bars (right bar of each pair) represent cells treated with 10 μg of the control molecule 4-methyl-piperazino-acetic acid ethyl ester in ethanol solution. The two bars on the left represent freshly isolated T cells and the two bars on the right represent transformed Jurkat cells.

FIGS. 4A-4I show that amino-phenyl-acetic acid octadec-(Z)-9-enyl ester in ethanol solution induced massive apoptosis of a transformed B cell line. Apoptosis was measured by FACS analysis with a hypo-diploid nuclei propidium iodide (PI) staining of an amino-phenyl-acetic acid octadec-(Z)-9-enyl ester treated p53 expressing L-12 mouse B cell line. FIGS. 4A-4C: L-12 cells treated with 0, 10 or 100 μg/ml of amino-phenyl-acetic acid octadec-(Z)-9-enyl ester in ethanol solution, respectively; FIGS. 4D-4F: L-12 cells treated with 0, 10 or 100 μg/ml of the control reagent 4-methyl-piperazino-acetic acid ethyl ester in ethanol solution, respectively; and FIGS. 4G-4I: L-12 cells treated with an ethanol volume corresponding to 0, 10 or 100 μg/ml of amino-phenyl-acetic acid octadec-(Z)-9-enyl ester, respectively. FL2-A is the measure of fluorescence. The % represents the percentage of cells in the sub-GO state.

FIGS. 5A-5I show that freshly isolated B cells treated with amino-phenyl-acetic acid octadec-(Z)-9-enyl ester in ethanol solution are partially protected from the spontaneous apoptosis. Apoptosis was measured by FACS analysis with a hypo-diploid nuclei propidium iodide (PI) staining of freshly isolated mouse B cells treated with amino-phenyl-acetic acid octadec-(Z)-9-enyl ester in ethanol solution. FIGS. 5A-5C: L-12 cells treated with 0, 1 or 100 μg/ml of amino-phenyl-acetic acid octadec-(Z)-9-enyl ester, respectively; FIGS. 5D-5F: L-12 cells treated with 0, 1 or 100 μg/ml of the control reagent 4-methyl-piperazino-acetic acid ethyl ester, respectively; and FIGS. 5G-5I: L-12 cells treated with an ethanol volume corresponding to 0, 1 or to 100 μg/ml of amino-phenyl-acetic acid octadec-(Z)-9-enyl ester in ethanol solution, respectively.

FIGS. 6A-6C show that amino-phenyl-acetic acid octadec-(Z)-9-enyl ester in ethanol solution inhibits lung tumor development and preserves life, and that intranasal administration is more effective than subcutaneous administration. Mice were injected intravenously with 500,000 cells of a virulent 3LL clone, D122, and treated daily for 35 days with either 5% ethanol solution (control); 100 μg amino-phenyl-acetic acid octadec-(Z)-9-enyl ester in 5% ethanol solution subcutaneously (SC); or 100 μg amino-phenyl-acetic acid octadec-(Z)-9-enyl ester in 5% ethanol solution intranasally (IN). The lung weight was measured for each mouse living at the end of the experiment. FIG. 6A: treatment with 5% ethanol solution administered subcutaneously without amino-phenyl-acetic acid octadec-(Z)-9-enyl ester; FIG. 6B: subcutaneous treatment with 100 μg of amino-phenyl-acetic acid octadec-(Z)-9-enyl ester in 5% ethanol solution; FIG. 6C: intranasal treatment with 100 μg/ml of amino-phenyl-acetic acid octadec-(Z)-9-enyl ester in 5% ethanol solution. A lung weight of 1500 represents dead mice.

FIGS. 7A-7D show that amino-phenyl-acetic acid octadec-(Z)-9-enyl ester in 5% ethanol solution administered intranasally, inhibits lung tumor development and preserves life and is more effective than amino-phenyl-acetic acid octadec-(Z)-9-enyl ester in PBS alone. Male mice were injected with a virulent 3LL clone, D122 and were either left untreated, or were treated daily for 30 days with intranasal administration of either 5% ethanol solution (control); 100 μg of amino-phenyl-acetic acid octadec-(Z)-9-enyl ester in PBS; or 100 μg of amino-phenyl-acetic acid octadec-(Z)-9-enyl ester in 5% ethanol solution. The lung weight was measured for each mouse living at the end of the experiment. FIG. 7A: no treatment; FIG. 7B: treatment with ethanol solution without amino-phenyl-acetic acid octadec-(Z)-9-enyl ester; FIG. 7C: treatment with 100 μg of amino-phenyl-acetic acid octadec-(Z)-9-enyl ester in PBS; FIG. 7D: treatment with 100 μg of amino-phenyl-acetic acid octadec-(Z)-9-enyl ester in 5% ethanol solution. A lung weight of 1500 represents dead mice.

### DETAILED DESCRIPTION OF THE INVENTION

Apoptosis, one of the best-studied forms of programmed cell death processes, plays an important role during the development and life-cycle of most multicellular organisms. The mechanisms underlying the initiation and manifestation of apoptotic cell death are the focus of the most recent cell death research. Generally, it is believed that cells are eliminated via a highly ordered and controlled program. This program might consist of the successive activation of unique apoptosis-specific genes, which are solely involved in the regulation of the programmed cell death. However, more and more evidence is accumulating that novel genes are not activated or induced during apoptosis, but rather many well-known genes previously described for their roles in processes such as proliferation and differentiation and belonging, for example, to the protein families of immediate-early genes and transcription factors become activated.

Additionally, it is now well known that failure of cells to undergo apoptosis is a common feature of many cancers, and that apoptosis and the genes that control it have a profound effect on the malignant phenotype. It is also well known that most cytotoxic anticancer agents induce apoptosis.

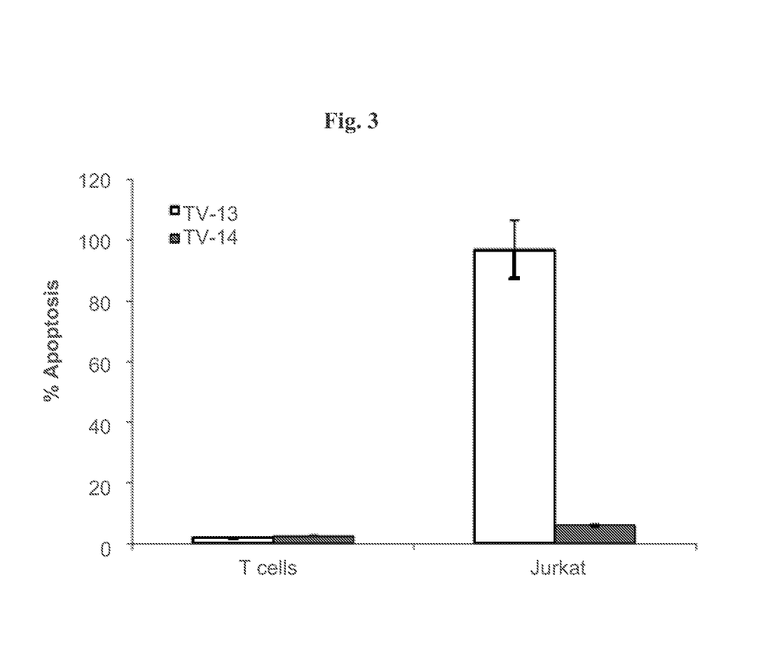

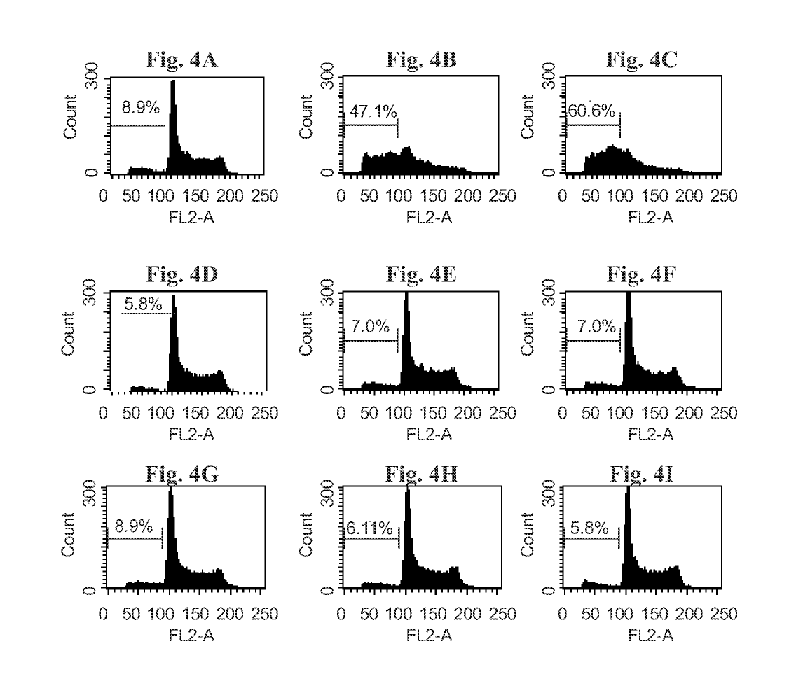

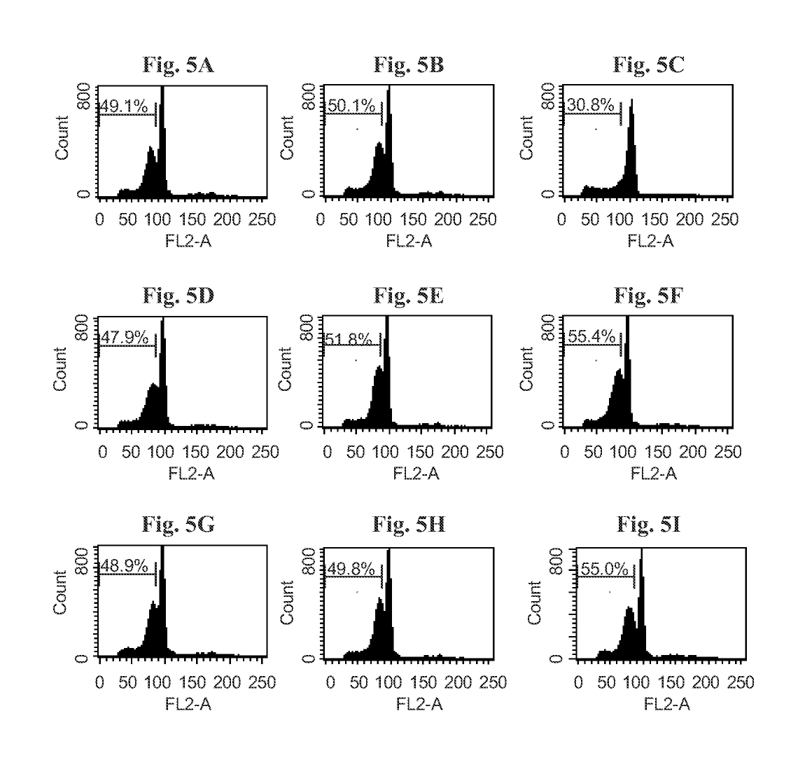

In a study mentioned in Example 3 hereinafter, we found that Jurkat tumor line cells were more sensitive than human peripheral blood T cells to apoptosis induced by amino-phenyl-acetic acid octadec-(Z)-9-enyl ester dissolved in ethanol solution (FIG. 3). We then proceeded to study the effect of amino-phenyl-acetic acid octadec-(Z)-9-enyl ester in ethanol solution in transformed B cells and found that amino-phenyl-acetic acid octadec-(Z)-9-enyl ester induced massive apoptosis of the transformed B cells while it partially protected freshly isolated B cells from apoptosis (see Example 3 and FIGS. 4-5).

Additionally, it has been found that amino-phenyl-acetic acid octadec-(Z)-9-enyl ester has anti-cancer activity both against lung tumor and against metastases also when not dissolved in ethanol solution (see Example 1).

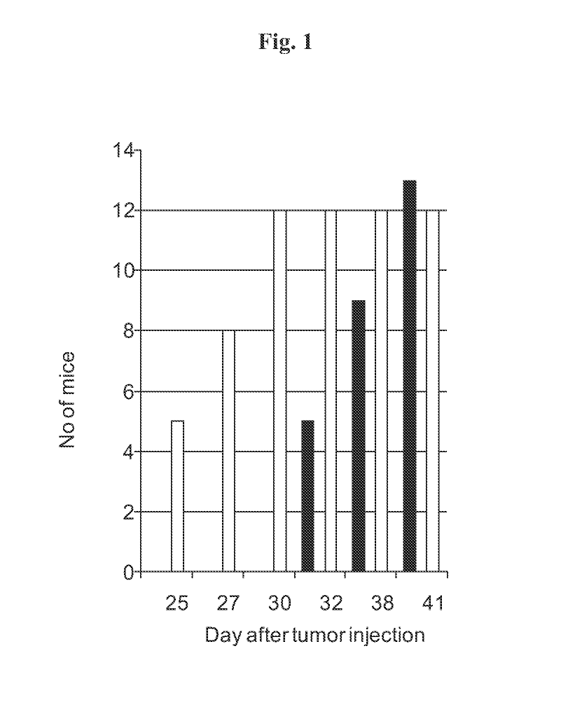

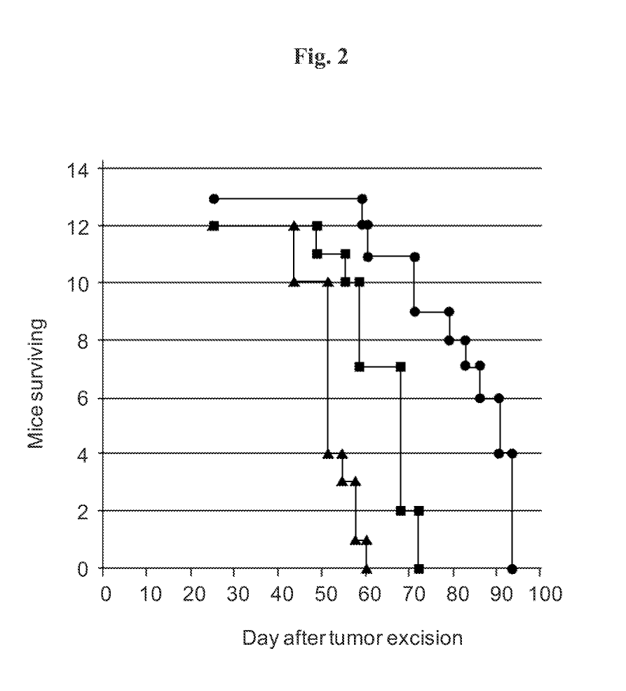

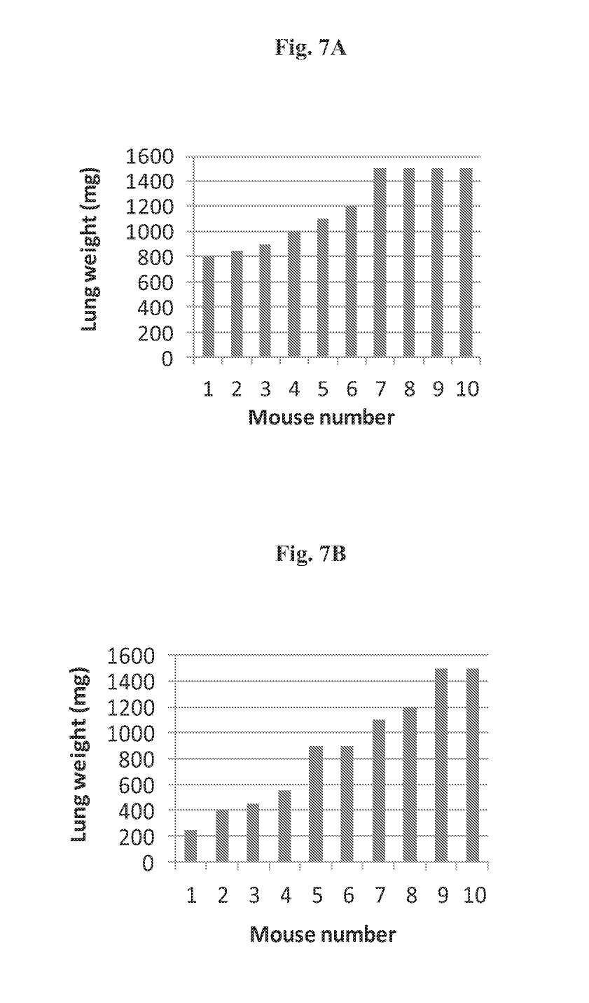

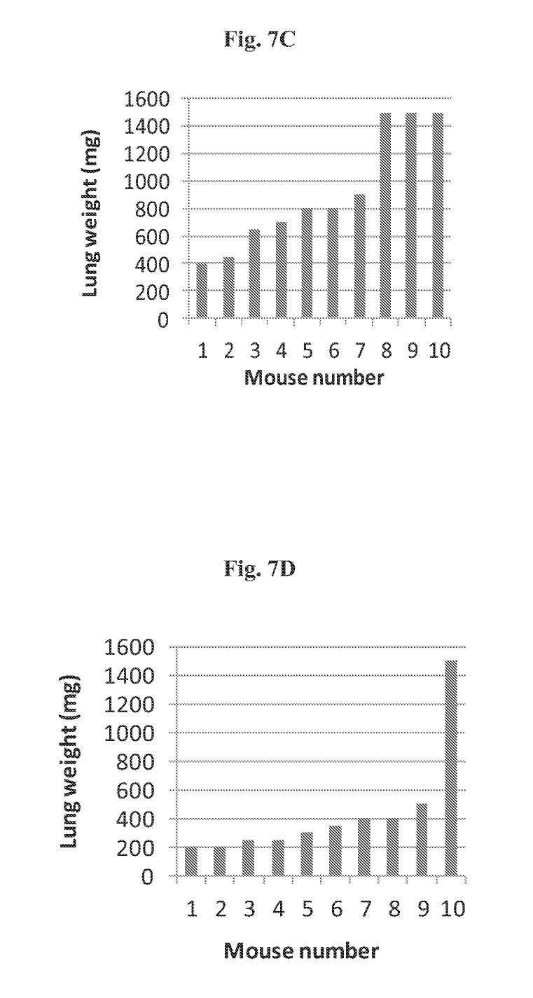

Next, we studied the effect of amino-phenyl-acetic acid octadec-(Z)-9-enyl ester both in aqueous vehicle and in ethanol solution on gene expression in unstimulated T cells and T cells activated by anti-CD3 antibody as described in Example 6 and Tables 1-8. In ethanol, we found down-regulation of genes that protect against apoptosis, such as BCL2 and insulin like growth factor 2 (see Tables 6 and 8) and up-regulation of genes that cause apoptosis, such as tumor necrosis factor receptor and cathepsin (see Tables 2, 3, 5 and 7). This effect was not seen in an aqueous vehicle. These results suggest that amino-phenyl-acetic acid octadec-(Z)-9-enyl ester exhibits different biological activity in an aqueous vehicle or in ethanol solution. This may cause their anticancer activity to be exerted through different pathways. The results show that amino-phenyl-acetic acid octadec-(Z)-9-enyl ester dissolved in an aqueous vehicle has anti-cancer activity (see Example 1, FIGS. 1 and 2), but it is a more effective apoptosis enhancing anti-cancer agent when dissolved in ethanol solution (Example 5, FIGS. 7C-7D).

The present invention relates to the compound amino-phenyl-acetic acid octadec-(Z)-9-enyl ester (Formula I), an enantiomer thereof or a pharmaceutically acceptable salt thereof, for use in treatment of tumors and metastases.

In certain embodiments, the compound is the racemic amino-phenyl-acetic acid octadec-(Z)-9-enyl ester. In certain embodiments, the enantiomer is (R)-amino-phenyl-acetic acid octadec-(Z)-9-enyl ester (Formula Ia). In certain embodiments, the enantiomer is (S)-amino-phenyl-acetic acid octadec-(Z)-9-enyl ester (Formula Ib).

In certain embodiments, the compound is an enantiomerically pure compound. In certain embodiments, the compound is an enantiomerically enriched compound.

“Enantioenriched compound” or “enantiomerically enriched compound” as used herein means a composition of a chiral substance whose enantiomeric ratio is greater than 50:50 but less than 100:0 of the specified enantiomer (See IUPAC Compendium of Chemical Terminology, “Goldbook”, Second Edition, 1997).

“Enantiopure compound” or “enantiomerically pure compound” as used herein means a composition containing molecules all having the same chirality sense (within the limits of detection). (See IUPAC Compendium of Chemical Terminology, “Goldbook”, Second Edition, 1997).

The amino-phenyl-acetic acid octadec-(Z)-9-enyl ester racemate can be synthesized as disclosed in WO 2004/03284, and its R and S enantiomers can be synthesized as disclosed in WO 2008/106092.

Examples of pharmaceutically acceptable salts include, but are not limited to, mineral or organic acid salts of the basic amino residues. The salts can be made using an organic or inorganic acid. Such acid salts include chlorides, bromides, sulfates, nitrates, phosphates, sulfonates, formates, tartrates, maleates, malates, citrates, benzoates, salicylates, ascorbates, and the like. The term “pharmaceutically acceptable salt” in this respect, refers to relatively non-toxic, salts of compounds used in the present invention.

In certain embodiments, the compound of formula I used in the invention, the enantiomer thereof or the pharmaceutically acceptable salt thereof is dissolved in an ethanol solution. In certain embodiments, the ethanol solution comprises from 4 to 20% or from 4 to 10% or from 5 to 10% ethanol in an aqueous vehicle. In certain embodiments, the ethanol solution comprises 5% ethanol in an aqueous vehicle.

Amino-phenyl-acetic acid octadec-(Z)-9-enyl ester is an ester of oleyl alcohol with D-phenyl alanine. It has a long hydrophobic segment with a hydrophilic head of an amine group which at physiological pH is positively charged. As such, it behaves like a typical micelle forming compound, with a critical micellar concentration in water. Such micelles disaggregate in the presence of alcohol. As shown in Example 2, at 10 μM concentration, amino-phenyl-acetic acid octadec-(Z)-9-enyl ester in PBS is in the form of micellar aggregates which disintegrate in the presence of above 4% ethanol. This disintegration of micellar aggregates may explain the different biological activity exhibited by amino-phenyl-acetic acid octadec-(Z)-9-enyl ester in PBS as compared with ethanol solution, as discussed above.

The terms “tumor” and “cancer” are herein used interchangeably.

In certain embodiments, the tumor to be treated according to the invention is selected from lung, brain, stomach, tongue, esophageal, colorectal, liver, gallbladder, pancreatic, renal, bladder, nasopharyngeal, laryngeal, skin, mammary, testicular, ovarian and uterus cancer, and metastases thereof. In certain embodiments, the tumor is a tumor metastasis. In a certain embodiment, the tumor is lung cancer or lung metastasis.

The present invention further relates to a pharmaceutical composition comprising amino-phenyl-acetic acid octadec-(Z)-9-enyl ester, an enantiomer thereof or a pharmaceutically acceptable salt thereof, and a pharmaceutically acceptable carrier, for the treatment of tumors and metastases.

The pharmaceutical composition provided by the present invention may be in solid, semisolid or liquid form and may further include pharmaceutically acceptable fillers, carriers or diluents, and other inert ingredients and excipients.

As used herein, a “pharmaceutically acceptable carrier” is a pharmaceutically acceptable solvent, suspending agent or vehicle, for delivering the instant compounds to the patient. The carrier may be liquid or solid and is selected with the planned manner of administration in mind. Liposomes are also a pharmaceutical carrier.

The composition can be formulated for administering by any suitable route such as, but not limited to, topical, oral, intranasal, or parenteral e.g. by injection through subcutaneous, intravenous, intramuscular, or any other suitable route. In certain embodiments, the pharmaceutical composition is formulated for administration intravenously, subcutaneously, intranasally, topically or orally.

By “topical administration” it is meant that the composition is applied to body surfaces, e.g. skin or mucous membranes such as nose, vagina, anus, throat, eyes and ears, and can be absorbed through mucous membranes, such as those in the gastrointestinal tract, e.g. stomach and colon, the urinary tract, e.g., kidney, urethra, bladder and prostate, the genital tract, e.g. uterus and cervix or the respiratory tract.

For topical administration, the active compounds used in the invention may be formulated as solutions, gels, ointments, creams, suspensions, etc. as are well-known in the art.

The active agent can be administered in the form of a tablet or capsule, liposome, as an agglomerated powder or in a liquid form. Examples of suitable solid carriers include lactose, sucrose, gelatin and agar. Capsule or tablets can be easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

For parenteral administration, the compound used in the invention may be formulated by mixing the compound at the desired degree of purity, in a unit dosage injectable form (solution, suspension, or emulsion), with a pharmaceutically acceptable carrier, i.e., one that is non-toxic to recipients at the dosages and concentrations employed and is compatible with other ingredients of the formulation. Generally, the formulations are prepared by contacting the compound uniformly and intimately with liquid carriers or finely divided solid carriers or both. Then, if necessary, the product is shaped into the desired formulation. Preferably, the carrier is a parenteral carrier, more preferably a solution that is isotonic with the blood of the recipient. Examples of such carrier vehicles include water, saline, Ringer's solution, and dextrose solution. Non-aqueous vehicles such as fixed oils can be also useful, as well as liposomes. These preparations can be made by conventional methods known to those skilled in the art, for example as described in “Remington's Pharmaceutical Science”, A. R. Gennaro, ed., 17th edition, 1985, Mack Publishing Company, Easton, Pa., USA.

The present invention still further relates to methods for treating a tumor in a subject in need thereof, comprising administering to said subject a therapeutically effective amount of amino-phenyl-acetic acid octadec-(Z)-9-enyl ester, an enantiomer thereof or a pharmaceutically acceptable salt thereof.

As used herein, the term “therapeutically effective amount” refers to the quantity of a component that is sufficient to yield a desired therapeutic response without undue adverse side effects (such as toxicity, irritation, or allergic response) commensurate with a reasonable benefit/risk ratio when used in the manner of this invention.

The dosage to be administered will depend on the state of the patient and severity of the disease and will be determined as deemed appropriate by the practitioner. According to certain embodiments, the dosage is between 0.05 and 2 mg/kg, or from 0.1 and 1 mg/kg. According to certain embodiments, the dosage is 0.3 mg/kg. According to certain embodiments, the dosage is from 5 to 100 mg per administration, or from 10 to 50 mg per administration, or from 20 to 40 mg per administration. According to certain embodiments, the dosage is 25 mg per administration.

The term “treating cancer” as used herein refers to the inhibition of the growth or causing death of cancer cells. Preferably such treatment also leads to the regression of tumor growth, i.e. to the decrease in size or complete regression of the tumor. In preferred embodiments, the term refers to treatment and alleviation or complete cure of disseminated tumors, namely, of metastases.

The present invention relates yet further to a method for enhancing apoptosis in a tumor or metastases, comprising administering to an individual in need thereof a therapeutically effective amount of amino-phenyl-acetic acid octadec-(Z)-9-enyl ester, an enantiomer thereof or a pharmaceutically acceptable salt thereof, dissolved in ethanol solution, thus enhancing apoptosis of said tumor or metastases in said individual.

In certain embodiments, the compounds used in the present invention may be administered together with other anti-cancer agents as known in the art.

The invention will now be illustrated by the following non-limiting Examples.

### Examples

### Materials and Methods

### Preparation of amino-phenyl-acetic acid octadec-(Z)-9-enyl ester for in vitro experiments

amino-phenyl-acetic acid octadec-(Z)-9-enyl ester was synthesized as disclosed in WO 2008/106092 (enantiomers) or in WO 2004/03284 (racemate) and dissolved in Phosphate buffered saline (PBS) without calcium and magnesium at a concentration of 1 mg/ml. The solution was incubated at 37° C. for a few minutes, and then vigorously vortexed immediately before use. The amino-phenyl-acetic acid octadec-(Z)-9-enyl ester in PBS solution was further diluted for use in culture medium to obtain the desired concentration; for example, a dilution of 1:100 (10 μl in 1 ml) in culture medium produced a final concentration of 10 jug/ml. Culture medium: RPMI 1640 containing antibiotics (1% Penicillin and 1% Streptavidin), 1% glutamine and 10% heat-inactivated Fetal calf serum (Hyclon Logan, Utah)

### Preparation of amino-phenyl-acetic acid octadec-(Z)-9-enyl ester in PBS for in vivo experiments

amino-phenyl-acetic acid octadec-(Z)-9-enyl ester was dissolved in PBS without calcium and magnesium at the stock concentrations indicated below. Each solution was vortexed and incubated at 50° C. for 5 minutes, and brought to room temperature, and vigorously vortexed again immediately before use. For subcutaneous injection or intranasal application the stock concentrations were 1, 2, 5 and 10 mg/ml and 100 μl were used, yielding 0.1, 0.2, 0.5 or 1 mg per mouse, respectively.

### Preparation of amino-phenyl-acetic acid octadec-(Z)-9-enyl ester in ethanol solution

amino-phenyl-acetic acid octadec-(Z)-9-enyl ester was dissolved in 100% ethanol at a concentration of 20 mg/ml; this stock solution was stored at −20 C for further use. For in vitro experiments, immediately before use, amino-phenyl-acetic acid octadec-(Z)-9-enyl ester in ethanol stock solution was diluted in culture medium at 1:20 to obtain a 5% ethanol solution of 1 mg/ml amino-phenyl-acetic acid octadec-(Z)-9-enyl ester in the culture medium; this solution was further diluted in culture medium to obtain the desired concentration for the in vitro test; for example, a dilution of 1:100 (10 μl in 1 ml) in culture medium produced a final concentration of 10 μg/ml for testing. For in vivo experiments, the 20 mg/ml stock was diluted in PBS to 1 mg/ml in 5% ethanol solution, and the appropriate volume was used.

Fluorescence Activated Cell Sorter (FACS) Analysis with a Hypo-Diploid Nuclei Propidium Iodide (PI) Staining:

B or T-cell apoptosis was detected by flow cytometry. Briefly, purified cells were seeded in 24 well plates, 5×10⁵ per well, and incubated for 48 h at 37° C. 5% CO₂ in the presence of HSP60 (heat shock protein 60) at the indicated concentration of amino-phenyl-acetic acid octadec-(Z)-9-enyl ester. For cell cycle analysis, the cells were washed once in cold PBS, fixed in 2 ml cold methanol (−20° C.) for 30 min, centrifuged and resuspended in 0.5 ml PBS containing RNase A (100 μg/ml) and propidium iodide (PI, 50 μg/ml). The cells were then subjected to flow cytometric analysis; for each sample 10,000 were collected and cell-cycle distribution was analyzed according to relative DNA content (PI staining). Cell debris was electronically gated out by their low FSC (Forward Scatter), and the percentage of cells in different cell cycle phases was computed using CellQuest software (Becton Dickinson, Mountainview, Calif.). The cells were then collected, stained by APO-DIRECT TUNNEL kit (Phnxflow, CITY, Calif., USA); mitochondrial potential was measured by DePsipher (R&D Systems, Minneapolis, Minn.) according to the manufacturer's procedure. Intracellular caspase activity was measured by Apostat kit (R&D Systems), according to the manufacturer's procedure. The cells were analyzed by Flow cytometry using FACSort (Becton Dickinson) and CellQuest software (Becton Dickinson).

Propidium Iodide (PI) and Annexin Staining:

the procedure was carried out essentially according to the Becton Dickinson protocol described at http://www.bdbiosciences.com/support/resources/protocols/annexin.jsp.

Briefly, cells were washed twice with cold PBS and then resuspended in 0.01 M HEPES, pH 7.4; 0.14 M NaCl; 2.5 mM CaCl₂ at a concentration of ˜1×10⁶ cells/ml. 100 μl of the solution (˜1×10⁵ cells) was transferred to a 5 ml culture tube and annexin V-FITC (BD cat. no. 556420, 556419) and 2 μl PI (BD Cat. no. 556463) were added. The cells were gently mixed and incubated for 15 minutes at room temperature in the dark. To each tube 400 μl of 0.01 M HEPES, pH 7.4; 0.14 M NaCl; 2.5 mM CaCl₂ were added and the tube was analyzed by flow cytometry.

Lung Carcinoma Mouse Model:

Clone D122 of the 3LL mouse Lewis Lung carcinoma, a standard model of growth and metastasis, was used. Syngeneic 8-weeks old C57BL/6 male mice were injected into one hind footpad with 50,000 3LL tumor cells. Local growth of the tumor was followed. Tumors that reached the size of 8 mm×8 mm were excised; tumor excision at this stage was known to trigger the growth of lung metastases that eventually killed the mice. This model allows for investigation of the effect of treatment on two phases of the tumor progression: 1) the effect on local growth, as determined by the time it takes for the tumors to reach the size for excision; and 2) the effect on tumor metastasis as measured by the time it takes for death to occur after excision.

Microarray Experiments:

CD3 positive T cells were extracted from a healthy donor according to a standard protocol, and seeded in T cell medium including RPMI, 10% fetal calf serum, sodium pyruvate, L-Glutamine, and Penicillin/Streptomycin. Following an overnight incubation with medium, the cells were treated by incubating for 2 hours at 37° C. with amino-phenyl-acetic acid octadec-(Z)-9-enyl ester at a concentration of 10 micrograms/ml in a final volume of 10 ml in T-cell medium with or without 0.05% ethanol. After incubation, half the cells from each treatment were centrifuged and 2 ml of Tri-reagent (Sigma) were added to the pellet. The other half of the cells from each treatment were transferred to anti-CD3 antibody coated 24 well plates for an overnight incubation at 37° C. Coating plates with an anti-CD3 antibody was carried out by incubating with 2 micrograms/ml of OKT3 anti-CD3 antibody overnight at 4° C., washing three times with PBS and blocking with filtered 1% Bovine Serum Albumin for 1 hour. After activation with anti-CD3 antibody, cells were centrifuged and 2 ml of Tri-reagent were added to the pellet.

RNA was extracted from unstimulated cells and from cells activated with anti-CD3 according to standard protocols and used for hybridization with a Human Genome U133A 2.0 (Affymetrix, Catalogue number 900468), according to the manufacturer's instructions. The results of the hybridization were read by a GeneChip scanner 3000 (Affymetrix) and analyzed by QuantArray (GSI lumonics).

### Example 1

### The in vivo effect on lung carcinoma of amino-phenyl-acetic acid octadec-(Z)-9-enyl ester in PBS administered subcutaneously in mice

Three groups of 12-13 mice each were injected with 3LL tumor cells as described in the Materials and Methods section, and were treated subcutaneously with 100 μg of amino-phenyl-acetic acid octadec-(Z)-9-enyl ester injected twice weekly (every Monday and Thursday) either from day 6 after tumor injection (to investigate the effect on local growth and metastasis) or from the time of excision of the tumor (to investigate the effect on metastasis only), or were injected with PBS as a control. FIG. 1 shows the effect of treatment with amino-phenyl-acetic acid octadec-(Z)-9-enyl ester on the day following tumor injection in which tumors reached a size of 8 mm×8 mm. Tumors were excised on the same day. Mice treated with PBS or mice treated with amino-phenyl-acetic acid octadec-(Z)-9-enyl ester in PBS only following tumor excision reached excision size beginning on day 25 (white bars). In contrast, mice treated with amino-phenyl-acetic acid octadec-(Z)-9-enyl ester from day 6 following injection (black bars) reached excision size on day 32, and all mice were excised by day 41. Thus, amino-phenyl-acetic acid octadec-(Z)-9-enyl ester twice weekly at the dose of 100 μg inhibited local tumor growth.

FIG. 2 shows the effect of treatment with amino-phenyl-acetic acid octadec-(Z)-9-enyl ester on mortality from lung metastasis, caused by the excision of the initial tumor. The PBS-treated mice reached 50% mortality on day 50 (triangles); the mice receiving amino-phenyl-acetic acid octadec-(Z)-9-enyl ester in PBS after tumor excision reached 50% mortality only on day 68 (squares); and the mice treated with amino-phenyl-acetic acid octadec-(Z)-9-enyl ester in PBS from day 6 following tumor implantation reached 50% mortality on day 90 (circles). Thus, amino-phenyl-acetic acid octadec-(Z)-9-enyl ester treatment is effective in prolonging survival even when administered following tumor excision; however, earlier treatment is more effective.

### Example 2

### Amino-phenyl-acetic acid octadec-(Z)-9-enyl ester aggregates disintegrate in an ethanol solution

Light scattering at 90 degrees can be used to assess the amount of aggregates in a solution. Higher readings are indicative of a higher degree of aggregation, while lower readings are indicative of disintegration of aggregates into monomers. A series of 10 μM amino-phenyl-acetic acid octadec-(Z)-9-enyl ester solutions was prepared in PBS containing ethanol concentrations of 0-20%. The intensity of light scattering of each solution was recorded at 90 degrees with a 560 nm beam in a Perkin Elmer fluorimeter. The results, presented in a relative scale with respect to the percent ethanol (% ethanol indicated in brackets), were as follows: 100 (0); 98 (0.5); 90 (1): 75 (2); 45(3); 26 (4); 10 (5); 8 (10); 6 (20). These results clearly indicate that at 10 μM concentration of amino-phenyl-acetic acid octadec-(Z)-9-enyl ester in PBS it is in the form of micellar aggregates which disintegrate in the presence of above 4% ethanol, as shown by the intensity of light scattering being much less than 50%.

### Example 3

### The in vitro effect of amino-phenyl-acetic acid octadec-(Z)-9-enyl ester in ethanol solution on tumor lines compared with healthy cells

Study of the effects of amino-phenyl-acetic acid octadec-(Z)-9-enyl ester on human peripheral blood T cells versus a human tumor-cell line (Jurkat), as seen in FIG. 3, showed that treatment with amino-phenyl-acetic acid octadec-(Z)-9-enyl ester in ethanol solution resulted in 100% apoptosis of the Jurkat tumor line cells, while treatment of healthy T cells resulted in no more apoptosis than with a control molecule.

We used the p53 expressing L-12 cell line as a model of a transformed B cell line to study the effect of amino-phenyl-acetic acid octadec-(Z)-9-enyl ester. The L-12 p53 cell line was incubated with two different concentrations of amino-phenyl-acetic acid octadec-(Z)-9-enyl ester (10 or 100 μg per ml) in a 5% ethanol solution prepared as described above in the Materials and Methods section. After 48 h, the cells were harvested and analyzed for apoptosis by hypo-diploid nuclei PI staining. FIGS. 4A-4I show that amino-phenyl-acetic acid octadec-(Z)-9-enyl ester induced massive apoptosis of the transformed B cell line (FIGS. 4A-4C), as can be seen from the increase in the number of cells in the sub-GO state, corresponding to fragmented DNA. The control reagent 4-methyl-piperazino-acetic acid ethyl ester (FIGS. 4D-4F) or the same volume of the solvent ethanol (FIGS. 4G-4I) had no effect on the cycle of this cell line. FIGS. 5A-5I show that freshly isolated B cells treated with amino-phenyl-acetic acid octadec-(Z)-9-enyl ester were partially protected from the spontaneous apoptosis occurring in B-cell cultures (FIGS. 5A-5C). The control reagent 4-methyl-piperazino-acetic acid ethyl ester (FIGS. 5D-5F) and the same volume of the solvent ethanol (FIGS. 5G-5I) had no effect on the primary B-cell cell-cycle.

These findings suggest that amino-phenyl-acetic acid octadec-(Z)-9-enyl ester and related molecules might have significant and specific anti-tumor effects.

### Example 4

### The effect of amino-phenyl-acetic acid octadec-(Z)-9-enyl ester administered subcutaneously or intranasally on 3LL induced lung carcinoma

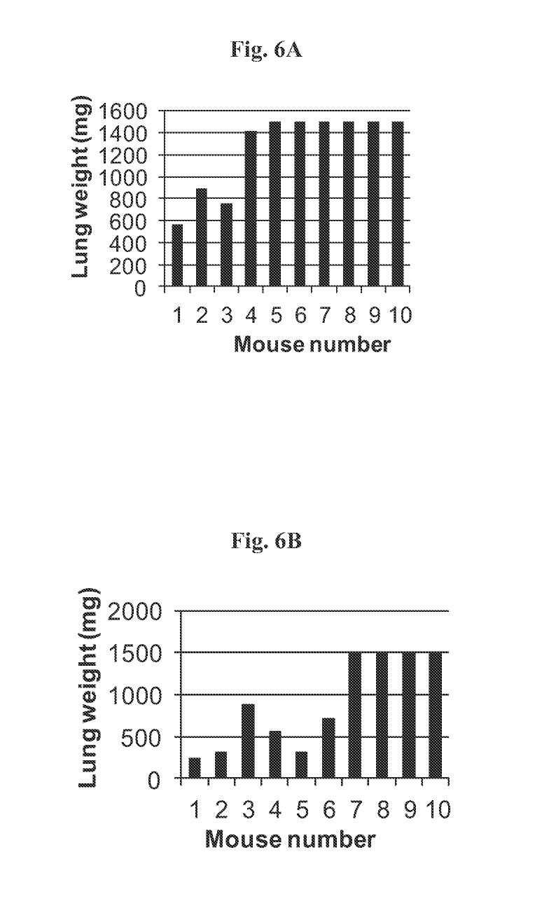

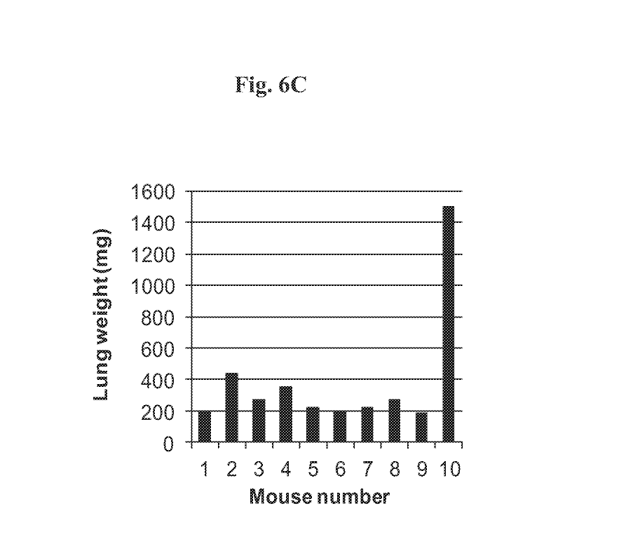

8-weeks old C57BL/6 male mice in groups of 10 were injected intravenously with 500,000 cells of a virulent 3LL clone, D122. Mice were treated daily for 35 days with either 5% ethanol in PBS (control); 100 μg amino-phenyl-acetic acid octadec-(Z)-9-enyl ester in 5% ethanol in PBS subcutaneously (SC); or 100 μg amino-phenyl-acetic acid octadec-(Z)-9-enyl ester in 5% ethanol in PBS intranasally (IN). As shown in FIGS. 6A-6C, while treatment with amino-phenyl-acetic acid octadec-(Z)-9-enyl ester subcutaneously reduced the number of dead mice from seven to four, as well as reducing the lung weight of the living mice from 903±364 to 501±252 (FIGS. 6A-6B), treatment with amino-phenyl-acetic acid octadec-(Z)-9-enyl ester intranasally reduced the number of dead mice to a single mouse and substantially reduced the lung weight of the living mice to 259±84 (FIG. 6C). In conclusion, 100 μg daily of amino-phenyl-acetic acid octadec-(Z)-9-enyl ester in 5% ethanol in PBS inhibits lung tumor development and preserves life, while intranasal administration was found to be more effective than subcutaneous administration.

### Example 5

### The effect of intranasal treatment with amino-phenyl-acetic acid octadec-(Z)-9-enyl ester in PBS or in ethanol solution on 3LL induced lung carcinoma

8-weeks old C57BL/6 male mice in groups of 10 were injected intravenously with 500,000 cells of a virulent 3LL clone, D122. Mice either left untreated, or were treated daily for 30 days with intranasal administration of either 5% ethanol in PBS (control); 100 μg of amino-phenyl-acetic acid octadec-(Z)-9-enyl ester in PBS; or 100 μg of amino-phenyl-acetic acid octadec-(Z)-9-enyl ester in 5% ethanol in PBS.

As shown in FIGS. 7A-7D, while treatment with amino-phenyl-acetic acid octadec-(Z)-9-enyl ester in PBS reduced the lung weight of the living mice from 718±351 in the control mice treated with 5% ethanol in PBS to 671±187 (FIGS. 7B-7C), treatment with amino-phenyl-acetic acid octadec-(Z)-9-enyl ester in ethanol solution reduced the lung weight of the living mice to 317±103 (FIG. 7D). In conclusion: 100 μg daily of intranasally administered amino-phenyl-acetic acid octadec-(Z)-9-enyl ester in ethanol solution inhibits lung tumor development and preserves life and is more effective than amino-phenyl-acetic acid octadec-(Z)-9-enyl ester in PBS alone.

### Example 6

### A comparison of the effect of amino-phenyl-acetic acid octadec-(Z)-9-enyl ester in an aqueous vehicle vs. in ethanol solution on gene regulation

The effects of amino-phenyl-acetic acid octadec-(Z)-9-enyl ester in ethanol solution or in an aqueous vehicle (hereinafter termed “in PBS”) on gene expression in unstimulated T cells and T cells activated by anti-CD3 antibody were compared by microarray analysis. As can be seen from Tables 1-8, the effects of amino-phenyl-acetic acid octadec-(Z)-9-enyl ester in ethanol solution are very different from those of amino-phenyl-acetic acid octadec-(Z)-9-enyl ester in PBS on both unstimulated and on activated T cells. This indicates that different mechanisms of action are used in the two cases, implying that they behave as two biologically different materials.

Unstimulated T cells treated with amino-phenyl-acetic acid octadec-(Z)-9-enyl ester in PBS resulted in no significantly up-regulated genes and several down-regulated genes, as seen in Table 1. In contrast, treating unstimulated cells with amino-phenyl-acetic acid octadec-(Z)-9-enyl ester in ethanol solution resulted in many up-regulated genes, as shown in Table 2, including the pro-apoptotic tumor necrosis factor receptor, and 573 down-regulated genes by 2-18 fold (data not shown). Table 3 shows genes that were up-regulated in cells treated with amino-phenyl-acetic acid octadec-(Z)-9-enyl ester in ethanol solution compared with amino-phenyl-acetic acid octadec-(Z)-9-enyl ester in PBS. 497 genes were down-regulated with amino-phenyl-acetic acid octadec-(Z)-9-enyl ester in ethanol solution compared with in PBS.

In activated T cells, treatment with amino-phenyl-acetic acid octadec-(Z)-9-enyl ester in PBS resulted in some up-regulated genes and no down-regulated genes (as shown in Table 4). In contrast, treatment with amino-phenyl-acetic acid octadec-(Z)-9-enyl ester in ethanol solution resulted in many up-regulated genes (shown in Table 5), including the pro-apoptotic cathepsin, and many down-regulated genes (shown in Table 6), including BCL2 and insulin like growth factor 2 that protect against apoptosis. Comparing Table 5 with Table 4 shows that different genes were up-regulated in either case. Tables 7 and 8 show a comparison of genes up-regulated or down-regulated, respectively, as a result of treatment with amino-phenyl-acetic acid octadec-(Z)-9-enyl ester in ethanol solution or in PBS.

These results suggest that amino-phenyl-acetic acid octadec-(Z)-9-enyl ester in PBS or in ethanol solution have different biological activities, and that when dissolved in ethanol solution, amino-phenyl-acetic acid octadec-(Z)-9-enyl ester is a more effective apoptosis enhancing anti-cancer agent than when dissolved in PBS.

The microarray data presented in the tables below can also be verified, if needed, by other methods for studying changes in gene expression such as northern blot, RT-PCR, and microarray with suitable probesets, such as described, inter alia, in M. Green and J. Sambrook, Molecular Cloning: A Laboratory Manual, 2012, CSHL Press.

TABLE 1
Down-regulated genes in unstimulated T cells treated with amino-phenyl-
acetic acid octadec-(Z)-9-enyl ester in PBS, compared with untreated
unstimulated T cells

| | | Gene | Fold |
|---|---|---|---|
| Probeset ID | Gene Title | Symbol | change |
| 206748_s_at | sperm associated antigen 9 | SPAG9 | −1.81 |
| 210784_x_at | leukocyte immunoglobulin- | LILRB2 /// | −1.84 |
| | like receptor, subfamily B (with | LILRB3 | |
| | TM and ITIM domains), | | |
| 206221_at | RAS p21 protein activator 3 | RASA3 | −2.23 |
| 214975_s_at | myotubularin related protein 1 | MTMR1 | −2.41 |

TABLE 2
Up-regulated genes in unstimulated T cells treated with amino-phenyl-acetic
acid octadec-(Z)-9-enyl ester in ethanol solution, compared with untreated
unstimulated T cells

| | | | Fold |
|---|---|---|---|
| Probeset ID | Gene Title | Gene Symbol | change |
| 204083_s_at | tropomyosin 2 (beta) | TPM2 | 2.39 |
| 218851_s_at | WD repeat domain 33 | WDR33 | 2.32 |
| 211709_s_at | C-type lectin domain family 11, member | CLEC11A | 2.19 |
| | A /// C-type lectin domain family 11, mem | | |
| 205781_at | chromosome 16 open reading frame 7 | C16orf7 | 2.15 |
| 214057_at | Myeloid cell leukemia sequence 1 (BCL2- | MCL1 | 2.12 |
| | related) | | |
| 222376_at | — | — | 2.11 |
| 44783_s_at | hairy/enhancer-of-split related with | HEY1 | 2.11 |
| | YRPW motif 1 | | |
| 206641_at | tumor necrosis factor receptor | TNFRSF17 | 2.06 |
| | superfamily, member 17 | | |
| 217817_at | actin related protein 2/3 complex, subunit | ARPC4 | 2.03 |
| | 4, 20 kDa | | |
| 210144_at | TBC1 domain family, member 22A | TBC1D22A | 2.03 |
| 218847_at | insulin-like growth factor 2 mRNA | IGF2BP2 | 2.02 |
| | binding protein 2 | | |
| 222285_at | immunoglobulin heavy constant delta | IGHD | 1.98 |
| 215450_at | — | — | 1.98 |
| 214370_at | S100 calcium binding protein A8 | S100A8 | 1.9 |
| 209381_x_at | splicing factor 3a, subunit 2, 66 kDa | SF3A2 | 1.95 |
| 218148_at | centromere protein T | CENPT | 1.94 |

TABLE 3
Up-regulated genes in unstimulated T cells treated with amino-phenyl-acetic
acid octadec-(Z)-9-enyl ester in ethanol solution compared with unstimulated T cells
treated with amino-phenyl-acetic acid octadec-(Z)-9-enyl ester in PBS

| | | | Fold |
|---|---|---|---|
| Probeset ID | Gene Title | Gene Symbol | change |
| 218851_s_at | WD repeat domain 33 | WDR33 | 2.79 |
| 205781_at | chromosome 16 open reading frame 7 | C16orf7 | 2.54 |
| 204802_at | Ras-related associated with diabetes | RRAD | 2.27 |
| 209263_x_at | tetraspanin 4 | TSPAN4 | 2.24 |
| 211709_s_at | C-type lectin domain family 11, member | CLEC11A | 2.10 |
| | A /// C-type lectin domain family 11, mem | | |
| 206641_at | tumor necrosis factor receptor superfamily, | TNFRSF17 | 2.08 |
| | member 17 | | |
| 213931_at | inhibitor of DNA binding 2, dominant | ID2 /// ID2B | 2.04 |
| | negative helix-loop-helix protein /// inhib | | |
| 32402_s_at | symplekin | SYMPK | 2.03 |
| 204718_at | EPH receptor B6 | EPHB6 | 2.019 |
| 210784_x_at | leukocyte immunoglobulin-like receptor, | LILRB2 /// | 2.00 |
| | subfamily B (with TM and ITIM domains), | LILRB3 | |
| 207159_x_at | CREB regulated transcription coactivator 1 | CRTC1 | 1.96 |
| 204695_at | cell division cycle 25 homolog A | CDC25A | 1.94 |
| | (S. cerevisiae) | | |
| 212563_at | block of proliferation 1 /// similar to block | BOP1 /// | 1.94 |
| | of proliferation 1 | LOC727967 | |
| 206548_at | hypothetical protein FLJ23556 | FLJ23556 | 1.94 |
| 218161_s_at | ceroid-lipofuscinosis, neuronal 6, late | CLN6 | 1.94 |
| | infantile, variant | | |

TABLE 4
Up-regulated genes in anti-CD3-activated T cells treated with amino-
phenyl-acetic acid octadec-(Z)-9-enyl ester in PBS compared with
untreated anti-CD3-activated T cells

| | | Gene | Fold |
|---|---|---|---|
| Probeset ID | Gene Title | Symbol | change |
| 206108_s_at | splicing factor, arginine/serine- | SFRS6 | 2.16 |
| | rich 6 | | |
| 208050_s_at | caspase 2, apoptosis-related | CASP2 | 2.04 |
| | cysteine peptidase (neural | | |
| | precursor cell expressed | | |
| 215101_s_at | chemokine (C-X-C motif) | CXCL5 | 1.995 |
| | ligand 5 | | |
| 205787_x_at | zinc finger CCCH-type | ZC3H11A | 1.95 |
| | containing 11A | | |
| 216563_at | Ankyrin repeat domain 12 | ANKRD12 | 1.94 |
| 221917_s_at | G-rich RNA sequence binding | GRSF1 | 1.92 |
| | factor 1 | | |
| 203294_s_at | lectin, mannose-binding, 1 | LMAN1 | 1.91 |

TABLE 5
Up-regulated genes in anti-CD3-activated T cells treated with amino-phenyl-
acetic acid octadec-(Z)-9-enyl ester in ethanol solution compared with untreated anti-
CD3-activated T cells

| | | | Fold |
|---|---|---|---|
| Probeset ID | Gene Title | Gene Symbol | change |
| 216598_s_at | chemokine (C-C motif) ligand 2 | CCL2 | 5.00 |
| 209278_s_at | tissue factor pathway inhibitor 2 | TFPI2 | 3.87 |
| 206214_at | phospholipase A2, group VII (platelet- | PLA2G7 | 3.44 |
| | activating factor acetylhydrolase, plasma) | | |
| 209395_at | chitinase 3-like 1 (cartilage glycoprotein- | CHI3L1 | 3.40 |
| | 39) | | |
| 209277_at | tissue factor pathway inhibitor 2 | TFPI2 | 3.32 |
| 204614_at | serpin peptidase inhibitor, clade B | SERPINB2 | 3.14 |
| | (ovalbumin), member 2 | | |
| 220322_at | interleukin 1 family, member 9 | IL1F9 | 3.10 |
| 206134_at | ADAM-like, decysin 1 | ADAMDEC1 | 2.87 |
| 219437_s_at | ankyrin repeat domain 11 | ANKRD11 | 2.84 |
| 203936_s_at | matrix metallopeptidase 9 (gelatinase B, | MMP9 | 2.74 |
| | 92 kDa gelatinase, 92 kDa type IV collage | | |
| 211506_s_at | interleukin 8 | IL8 | 2.69 |
| 209396_s_at | chitinase 3-like 1 (cartilage glycoprotein- | CHI3L1 | 2.58 |
| | 39) | | |
| 216563_at | Ankyrin repeat domain 12 | ANKRD12 | 2.48 |
| 210029_at | indoleamine-pyrrole 2,3 dioxygenase | INDO | 2.47 |
| 210943_s_at | lysosomal trafficking regulator | LYST | 2.45 |
| 215284_at | Sorting nexin 9 | SNX9 | 2.44 |
| 203510_at | met proto-oncogene (hepatocyte growth | MET | 2.35 |
| | factor receptor) | | |
| 205003_at | dedicator of cytokinesis 4 | DOCK4 | 2.33 |
| 204475_at | matrix metallopeptidase 1 (interstitial | MMP1 | 2.32 |
| | collagenase) | | |
| 215101_s_at | chemokine (C-X-C motif) ligand 5 | CXCL5 | 2.31 |
| 216575_at | — | — | 2.31 |
| 202087_s_at | cathepsin L | CTSL | 2.30 |
| 215967_s_at | lymphocyte antigen 9 | LY9 | 2.2 |
| 213797_at | radical S-adenosyl methionine domain | RSAD2 | 2.28 |
| | containing 2 | | |
| 205568_at | aquaporin 9 | AQP9 | 2.27 |
| 202917_s_at | S100 calcium binding protein A8 | S100A8 | 2.24 |
| 214038_at | chemokine (C-C motif) ligand 8 | CCL8 | 2.23 |
| 205184_at | guanine nucleotide binding protein (G | GNG4 | 2.23 |
| | protein), gamma 4 | | |
| 218035_s_at | RNA-binding protein | FLJ20273 | 2.17 |
| 207442_at | colony stimulating factor 3 (granulocyte) | CSF3 | 2.14 |
| 208018_s_at | hemopoietic cell kinase | HCK | 2.14 |
| 202833_s_at | serpin peptidase inhibitor, clade A (alpha-1 | SERPINA1 | 2.12 |
| | antiproteinase, antitrypsin), membe | | |
| 215415_s_at | lysosomal trafficking regulator | LYST | 2.09 |
| 204588_s_at | solute carrier family 7 (cationic amino acid | SLC7A7 | 2.07 |
| | transporter, y+ system), member 7 | | |
| 211429_s_at | serpin peptidase inhibitor, clade A (alpha-1 | SERPINA1 | 2.06 |
| | antiproteinase, antitrypsin), membe | | |
| 205067_at | interleukin 1, beta | IL1B | 2.03 |
| 208605_s_at | neurotrophic tyrosine kinase, receptor, type | NTRK1 | 2.03 |
| | 1 | | |
| 39402_at | interleukin 1, beta | IL1B | 2.03 |
| 202436_s_at | cytochrome P450, family 1, subfamily B, | CYP1B1 | 2.02 |
| | polypeptide 1 | | |
| 210845_s_at | plasminogen activator, urokinase receptor | PLAUR | 2.02 |
| 210118_s_at | interleukin 1, alpha | IL1A | 2.02 |
| 213975_s_at | lysozyme (renal amyloidosis) /// riboflavin | LYZ /// RFK | 2.00 |
| | kinase | | |
| 210145_at | phospholipase A2, group IVA (cytosolic, | PLA2G4A | 2.00 |
| | calcium-dependent) | | |
| 210772_at | formyl peptide receptor-like 1 /// formyl | FPRL1 | 2.00 |
| | peptide receptor-like 1 | | |
| 216243_s_at | interleukin 1 receptor antagonist | IL1RN | 1.99 |
| 208075_s_at | chemokine (C-C motif) ligand 7 /// | CCL7 | 1.99126 |
| | chemokine (C-C motif) ligand 7 | | |
| 206421_s_at | serpin peptidase inhibitor, clade B | SERPINB7 | 1.99 |
| | (ovalbumin), member 7 | | |
| 212657_s_at | interleukin 1 receptor antagonist | IL1RN | 1.98 |
| 222330_at | Phosphodiesterase 3B, cGMP-inhibited | PDE3B | 1.97 |
| 206569_at | interleukin 24 | IL24 | 1.97 |
| 210511_s_at | inhibin, beta A (activin A, activin AB alpha | INHBA | 1.96 |
| | polypeptide) | | |
| 205207_at | interleukin 6 (interferon, beta 2) | IL6 | 1.95 |
| 215223_s_at | superoxide dismutase 2, mitochondrial | SOD2 | 1.95 |
| 201109_s_at | thrombospondin 1 | THBS1 | 1.94 |
| 204232_at | Fc fragment of IgE, high affinity I, receptor | FCER1G | 1.94 |
| | for; gamma polypeptide | | |
| 217678_at | solute carrier family 7, (cationic amino acid | SLC7A11 | 1.92 |
| | transporter, y+ system) member 11 | | |
| 206025_s_at | tumor necrosis factor, alpha-induced protein | TNFAIP6 | 1.92 |
| | 6 | | |
| 203695_s_at | deafness, autosomal dominant 5 | DFNA5 | 1.92 |
| 203963_at | carbonic anhydrase XII | CA12 | 1.91 |
| 211924_s_at | plasminogen activator, urokinase receptor /// | PLAUR | 1.91 |
| | plasminogen activator, urokinase r | | |
| 212659_s_at | interleukin 1 receptor antagonist | IL1RN | 1.90 |

TABLE 6
Down-regulated genes in anti-CD3-activated T cells treated with amino-
phenyl-acetic acid octadec-(Z)-9-enyl ester in ethanol solution compared with
untreated anti-CD3-activated T cells

| | | | Fold |
|---|---|---|---|
| Probeset ID | Gene Title | Gene Symbol | change |
| 205296_at | — | — | −2.02 |
| 220651_s_at | MCM10 minichromosome maintenance | MCM10 | −2.05 |
| | deficient 10 (S. cerevisiae) | | |
| 205970_at | metallothionein 3 (growth inhibitory factor | MT3 | −2.05 |
| | (neurotrophic)) | | |
| 201438_at | collagen, type VI, alpha 3 | COL6A3 | −2.06 |
| 205024_s_at | RAD51 homolog (RecA homolog, E. coli) | RAD51 | −2.09 |
| | (S. cerevisiae) | | |
| 204567_s_at | ATP-binding cassette, sub-family G | ABCG1 | −2.12 |
| | (WHITE), member 1 | | |
| 218507_at | hypoxia-inducible protein 2 | HIG2 | −2.17 |
| 202409_at | insulin-like growth factor 2 (somatomedin | IGF2 /// INS- | −2.17 |
| | A) /// insulin- insulin-like growth fa | IGF2 | |
| 204603_at | exonuclease 1 | EXO1 | −2.25 |
| 204347_at | similar to Adenylate kinase isoenzyme 4, | LOC645619 /// | −2.25 |
| | mitochondrial (ATP-AMP transphosphoryla | LOC731007 | |
| 221478_at | BCL2/adenovirus E1B 19 kDa interacting | BNIP3L | −2.28 |
| | protein 3-like /// BCL2/adenovirus E1B 19k | | |
| 219670_at | chromosome 1 open reading frame 165 | C1orf165 | −2.29 |
| 204822_at | TTK protein kinase | TTK | −2.40 |
| 221165_s_at | interleukin 22 | IL22 | −2.63 |
| 201848_s_at | BCL2/adenovirus E1B 19 kDa interacting | BNIP3 | −2.99 |
| | protein 3 | | |
| 204348_s_at | adenylate kinase 3-like 1 | AK3L1 | −3.14 |
| 201849_at | BCL2/adenovirus E1B 19 kDa interacting | BNIP3 | −3.24 |
| | protein 3 | | |
| 202718_at | insulin-like growth factor binding protein 2, | IGFBP2 | −5.13 |
| | 36 kDa | | |

TABLE 7
Up-regulated genes in anti-CD3-activated T cells treated with amino-phenyl-
acetic acid octadec-(Z)-9-enyl ester in ethanol solution compared with anti-CD3-
activated T cells treated with amino-phenyl-acetic acid octadec-(Z)-9-enyl ester in
PBS

| | | | Fold |
|---|---|---|---|
| Probeset ID | Gene Title | Gene Symbol | Change |
| 216598_s_at | chemokine (C-C motif) ligand 2 | CCL2 | 5.27 |
| 213797_at | radical S-adenosyl methionine domain | | |
| | containing 2 | RSAD2 | 3.11 |
| 209278_s_at | tissue factor pathway inhibitor 2 | TFPI2 | 2.99 |
| 203510_at | met proto-oncogene (hepatocyte growth | MET | 2.76 |
| | factor receptor) | | |
| 220322_at | interleukin 1 family, member 9 | IL1F9 | 2.74 |
| 209395_at | chitinase 3-like 1 (cartilage glycoprotein- | CHI3L1 | 2.69 |
| | 39) | | |
| 216575_at | — | — | 2.69 |
| 203936_s_at | matrix metallopeptidase 9 (gelatinase B, | MMP9 | 2.67 |
| | 92 kDa gelatinase, 92 kDa type IV collage | | |
| 204614_at | serpin peptidase inhibitor, clade B | SERPINB2 | 2.60 |
| | (ovalbumin), member 2 | | |
| 209277_at | tissue factor pathway inhibitor 2 | TFPI2 | 2.53 |
| 206134_at | ADAM-like, decysin 1 | ADAMDEC1 | 2.41 |
| 214038_at | chemokine (C-C motif) ligand 8 | CCL8 | 2.33 |
| 210370_s_at | lymphocyte antigen 9 | LY9 | 2.25 |
| 202087_s_at | cathepsin L | CTSL | 2.18 |
| 209396_s_at | chitinase 3-like 1 (cartilage glycoprotein- | CHI3L1 | 2.15 |
| | 39) | | |
| 206214_at | phospholipase A2, group VII (platelet- | PLA2G7 | 2.12 |
| | activating factor acetylhydrolase, plasma) | | |
| 202917_s_at | S100 calcium binding protein A8 | S100A8 | 2.11 |
| 204508_s_at | carbonic anhydrase XII | CA12 | 2.09 |
| 210029_at | indoleamine-pyrrole 2,3 dioxygenase | INDO | 2.08 |
| 215967_s_at | lymphocyte antigen 9 | LY9 | 2.05 |
| 204588_s_at | solute carrier family 7 (cationic amino acid | SLC7A7 | 2.05 |
| | transporter, y+ system), member 7 | | |
| 205184_at | guanine nucleotide binding protein (G | GNG4 | 2.04 |
| | protein), gamma 4 | | |
| 203963_at | carbonic anhydrase XII | CA12 | 2.02 |
| 213975_s_at | lysozyme (renal amyloidosis) /// riboflavin | LYZ /// RFK | 2.02 |
| | kinase | | |
| 210845_s_at | plasminogen activator, urokinase receptor | PLAUR | 2.01 |
| 208075_s_at | chemokine (C-C motif) ligand 7 /// | CCL7 | 2.01 |
| | chemokine (C-C motif) ligand 7 | | |
| 217853_at | tensin 3 | TNS3 | 2.00 |
| 222330_at | Phosphodiesterase 3B, cGMP-inhibited | PDE3B | 1.99 |
| 202833_s_at | serpin peptidase inhibitor, clade A (alpha-1 | SERPINA1 | 1.97 |
| | antiproteinase, antitrypsin), membe | | |
| 208018_s_at | hemopoietic cell kinase | HCK | 1.93 |

TABLE 8
Down-regulated genes in anti-CD3-activated T cells treated with amino-
phenyl-acetic acid octadec-(Z)-9-enyl ester in ethanol solution compared with anti
CD3-activated T cells treated with amino-phenyl-acetic acid octadec-(Z)-9-enyl
ester in PBS

| | | | Fold |
|---|---|---|---|
| Probeset ID | Gene Title | Gene Symbol | Change |
| 203504_s_at | ATP-binding cassette, sub-family A (ABC1), | ABCA1 | −1.90 |
| | member 1 | | |
| 210117_at | sperm associated antigen 1 | SPAG1 | −1.90 |
| 203282_at | glucan (1,4-alpha-), branching enzyme 1 | GBE1 | −1.92 |
| | (glycogen branching enzyme, Andersen dis | | |
| 218585_s_at | denticleless homolog (Drosophila) | DTL | −1.93 |
| 201123_s_at | eukaryotic translation initiation factor 5A | EIF5A | −1.97 |
| 212141_at | MCM4 minichromosome maintenance | MCM4 | −1.98 |
| | deficient 4 (S. cerevisiae) | | |
| 221521_s_at | GINS complex subunit 2 (Psf2 homolog) | GINS2 | −1.99 |
| 221479_s_at | BCL2/adenovirus E1B 19 kDa interacting | BNIP3L | −2.00 |
| | protein 3-like /// BCL2/adenovirus E1B 19k | | |
| 203967_at | cell division cycle 6 homolog (S. cerevisiae) | CDC6 | −2.03 |
| 204822_at | TTK protein kinase | TTK | −2.04 |
| 201438_at | collagen, type VI, alpha 3 | COL6A3 | −2.16 |
| 207543_s_at | procollagen-proline, 2-oxoglutarate 4- | P4HA1 | −2.20 |
| | dioxygenase (proline 4-hydroxylase), alpha | | |
| 219670_at | chromosome 1 open reading frame 165 | C1orf165 | −2.22 |
| 218507_at | hypoxia-inducible protein 2 | HIG2 | −2.31 |
| 205519_at | WD repeat domain 76 | WDR76 | −2.42 |
| 202409_at | insulin-like growth factor 2 (somatomedin | IGF2 /// INS- | −2.42 |
| | A) /// insulin- insulin-like growth fa | IGF2 | |
| 221478_at | BCL2/adenovirus E1B 19 kDa interacting | BNIP3L | −2.45 |
| | protein 3-like /// BCL2/adenovirus E1B 19k | | |
| 204347_at | similar to Adenylate kinase isoenzyme 4, | LOC645619 /// | −2.52 |
| | mitochondrial (ATP-AMP transphosphoryla | LOC731007 | |
| 212142_at | MCM4 minichromosome maintenance | MCM4 | −2.80 |
| | deficient 4 (S. cerevisiae) | | |
| 221165_s_at | interleukin 22 | IL22 | −3.03 |
| 201849_at | BCL2/adenovirus E1B 19 kDa interacting | BNIP3 | −3.34 |
| | protein 3 | | |
| 201848_s_at | BCL2/adenovirus E1B 19 kDa interacting | BNIP3 | −3.39 |
| | protein 3 | | |
| 204348_s_at | adenylate kinase 3-like 1 | AK3L1 | −3.93 |
| 202718_at | insulin-like growth factor binding protein 2, | IGFBP2 | −6.02 |
| | 36 kDa | | |

1-13. (canceled)

14. A method for treating a tumor or a metastasis in a subject in need thereof, comprising administering to said subject a therapeutically effective amount of a compound comprising an amino-phenyl-acetic acid octadec-(Z)-9-enyl ester, an enantiomer thereof or a pharmaceutically acceptable salt thereof.

15. The method according to claim 14, wherein said compound, enantiomer thereof or pharmaceutically acceptable salt thereof is dissolved in ethanol solution.

16. A method for enhancing apoptosis in a tumor or a metastasis, comprising administering to an individual in need thereof a therapeutically effective amount of a compound comprising an amino-phenyl-acetic acid octadec-(Z)-9-enyl ester, an enantiomer thereof or a pharmaceutically acceptable salt thereof.

17. The method according to claim 14, wherein said compound is a racemic amino-phenyl-acetic acid octadec-(Z)-9-enyl ester.

18. The method according to claim 14, wherein said enantiomer is (R)-amino-phenyl-acetic acid octadec-(Z)-9-enyl ester or (S)-amino-phenyl-acetic acid octadec-(Z)-9-enyl ester.

19. The method according to claim 15, wherein said ethanol solution comprises from 4% to 20%, from 4% to 10% or from 5% to 10% ethanol in an aqueous vehicle.

20. The method according to claim 14, wherein said tumor is in lung, brain, stomach, tongue, esophagus, colon, rectum, liver, gallbladder, pancreas, kidney, bladder, pharynx, larynx, skin, mammary gland, testicle, ovary or uterus.

21. The method according to claim 14, wherein said tumor is in a lung.

22. The method according to claim 14, wherein said administering is intravenous, subcutaneous, intranasal, topical or oral.

23. The method according to claim 22, wherein said topical administering is to a mucous membrane.

24. The method according to claim 23, wherein said mucous membrane is in an intestinal tract, urinary tract, genital tract or respiratory tract.

25. The method according to claim 16, wherein said compound, enantiomer thereof or pharmaceutically acceptable salt thereof is dissolved in ethanol solution.

26. The method according to claim 16, wherein said compound is a racemic amino-phenyl-acetic acid octadec-(Z)-9-enyl ester.

27. The method according to claim 16, wherein said enantiomer is (R)-amino-phenyl-acetic acid octadec-(Z)-9-enyl ester or (S)-amino-phenyl-acetic acid octadec-(Z)-9-enyl ester.

28. The method according to claim 25, wherein said ethanol solution comprises from 4% to 20%, from 4% to 10% or from 5% to 10% ethanol in an aqueous vehicle.

29. The method according to claim 16, wherein said tumor is in lung, brain, stomach, tongue, esophagus, colon, rectum, liver, gallbladder, pancreas, kidney, bladder, pharynx, larynx, skin, mammary gland, testicle, ovary or uterus.

30. The method according to claim 16, wherein said tumor is in a lung.

31. The method according to claim 16, wherein said administering is intravenous, subcutaneous, intranasal, topical or oral.

32. The method according to claim 31, wherein said topical administering is to a mucous membrane.

33. The method according to claim 32, wherein said mucous membrane is in an intestinal tract, urinary tract, genital tract or respiratory tract.
